(19) United States
(12) Patent Application Publication
(10) Pub. No.: US 2026/0317804 A1
(43) Pub. Date: Oct. 15, 2026

(54) ENZYME-CATALYZED REGIOSELECTIVE POLYMERIZATION FOR PREPARATION OF LINEAR POLYLYSINE OR POLYARGININE

(71) Applicants: BASF SE, Ludwigshafen am Rhein (DE); KYOTO UNIVERSITY, Kyoto (JP)

(72) Inventors: Alexandros LAMPROU, Shanghai (CN); Klaus DITRICH, Goennheim (DE); Keiji NUMATA, Saitama (JP); Kousuke TSUCHIYA, Kyoto (JP); Kayo TERADA, Nishikyo-ku (JP)

(21) Appl. No.: 19/483,661

(22) PCT Filed: May 24, 2024
(86) PCT No.: PCT/CN2024/095205

§ 371 Date: Nov. 12, 2025

(30) Foreign Application Priority Data
May 26, 2023 (WO) PCT/CN2023/096588

(51) Int. Cl. C12P 21/02; C08G 69/04

(52) U.S. Cl. CPC C12P 21/02; C08G 69/04

(57) ABSTRACT

Disclosed herein is a process for preparation of linear polylysine or polyarginine by chemoenzymatic bulk polymerization of lysine ester or arginine ester respectively. Also disclosed herein is a method for controlling regioselectivity of polylysine or polyarginine in the chemoenzymatic bulk polymerization of lysine ester or arginine ester.

### TECHNICAL FIELD

The invention relates to a process for preparation of linear polylysine or polyarginine by chemoenzymatic bulk polymerization of lysine ester or arginine ester respectively. The present invention also relates to a method for controlling regioselectivity of polylysine or polyarginine in the chemoenzymatic bulk polymerization of lysine ester or arginine ester.

### BACKGROUND

In the past several decades, polylysines and derivatives have been investigated in many fields, for example, as preservatives in food and personal care products, as surfactants and moisturizing agents in personal care and household products, as nutrition sources in nutritional supplement products, and also as biologically active ingredients in cosmetic or dermatological products. Among various methods for synthesis of polylysines, the enzyme-catalyzed polymerization method attracted increasing interests, due to its advantages in milder reaction conditions, fewer side reactions, higher selectivity, environmental friendliness, and simpler operations without the tedious protection-deprotection.

Enzyme-catalyzed polymerization for preparation of polylysines were generally carried out in a mono-phase or multi-phase system using protease in the presence of water. It was believed that protease-catalyzed synthesis of water insoluble amino acid oligomers in aqueous media is driven by precipitation. The synthesis of water-soluble oligomers of amino acids, such as lysine can be controlled only in mixed phase systems where the equilibria is shifted in favor of the synthesis of polypeptides due to enhanced partitioning of peptide in the organic phase.

US2006/0252134A1 describes a process for chemoenzymatic polymerization of hydroxy carboxylic acids and amino acids. Among others, oligolysine is prepared from commercially available lysine ethyl ester dihydrochloride as substrate for papain. Multi-phase systems are described, namely a two-phase system comprising an aqueous papain suspension and toluene containing the substrate, a reverse micellar system comprising an aqueous papain solution and a reverse micellar solution of the substrate in isooctane, and a three-phase system comprising an aqueous papain suspension and the substrate in another two phases of octane and decafluoropentane. The oligolysine as obtained has degrees of polymerization between 2 and 9 with a peak maximum of 4. It is implied that the alpha group is only reactive, at least for the case of lysine.

FR2708938A1 describes a chemoenzymatic process for preparation of oligomers of L-lysine utilizing lysine ethyl ester dihydrochloride and trypsin. The polymerization is carried out in a water-isopropanol mixture until two or more phases are formed. The degree of polymerization ranges between 2 and 8 with a peak maximum of 3.3, while regioselectivity of the product is not discussed.

CN112226469A describes a chemoenzymatic process for preparation of oligomers of L-lysine utilizing commercially available lysine methyl ester dihydrochloride and trypsin, papain or alkaline protease in a solvent mixture of aqueous buffer with a pH of 6.5 to 8 and dimethyl sulfoxide. The degree of polymerization or regioselectivity of the obtained oligopeptides is not discussed.

CN110669810A describes a chemoenzymatic process for preparation of oligomers of L-lysine utilizing commercially available lysine methyl ester dihydrochloride with crosslinked bromelain, papain or neutral protease in a solvent mixture composed of aqueous buffer with a pH of 5.5 to 8 and t-butanol. The degree of polymerization or regioselectivity of the obtained oligopeptides is not discussed.

CN112301081A describes a chemoenzymatic process for preparation of oligomers of L-lysine utilizing commercially available lysine methyl ester dihydrochloride with trypsin in an aqueous buffer having a pH of 7 to 8.5. The degree of polymerization or regioselectivity of the obtained oligopeptides is not discussed.

Srinivasan et al. report papain-catalyzed preparation of oligopeptides in Papain Catalyzed Oligomerization in Monophasic Aqueous Organic Media—Synthesis and Characterization of Neutral and Polar Amino Acid Oligomers, Enz. Eng 2017, 6:161. Among others, oligolysine is obtained utilizing commercially available lysine ethyl ester dihydrochloride in a monophasic water-acetonitrile system. The degree of polymerization ranges between 2 and 10 with a peak maximum of 5, while regioselectivity is not discussed.

Aso et al. report preparation of oligolysine in Trypsin-catalyzed Oligomerization of L-Lysine Esters, Bioscience, Biotechnology, 56:5, 755-758 (1992). The preparation utilizes commercially available lysine ethyl, n-butyl or n-hexyl ester dihydrochloride in an aqueous solution at pH 10 in presence of NaCl. The degree of polymerization ranges between 2 and 8 with a peak maximum of 4, while the discussion indicates the α-amino group of lysine interacts with the enzyme.

Puigserver et al. report papain-catalyzed preparation of oligolysine in Papain-catalyzed Polymerization of Amino Acids in Low Water Organic Solvents, Biotechnology Letter, Vol. 13, No. 13, 161-166 (1991). The preparation utilizes lysine methyl or ethyl ester dihydrochloride prepared from lysine in the respective alcohol solution with thionyl chloride, and Papain modified with polyethylene glycol. The polymerization is carried out in acetonitrile with a small amount of water. The degree of lysine polymerization ranges between 2 and 12 with a peak maximum between 4 and 6, while regioselectivity is not discussed.

Gross et al. report protease-catalyzed preparation of oligolysine in Protease-Catalyzed Oligomerization of Commercially Available L-Lysine Ethyl Ester in Aqueous Solution, ACS Catalysis, 2011, 1, 9, 1022-1034. The preparation is carried out utilizing commercially available L-lysine ethyl ester dihydrochloride with bromelain, trypsin, chymotrypsin or papain as the protease in an aqueous buffer. The degree of polymerization may be up to 12 with a peak maximum of 3.6. With all used proteases, oligo-α-lysine is obtained.

Heretofore, preparation of polylysine through enzyme-catalyzed polymerization in bulk has never been reported. Chemoenzymatic polymerizations of amino acids in bulk was reported only for aspartic acid ester as the substrate. For example, Matsumura et al. report the polymerization of diethyl aspartate in bulk using a commercially available alkalophilic proteinase in Enzyme-catalyzed Polymerization of L-Aspartate, Macromol. Rapid Commun. 20, 7-11 (1999). Poly-α-aspartate is obtained with up to 88% α-linkages and up to 23 repeating units. The polymer is saponified to give polyaspartic acid sodium salt. Zhang et al. report bulk polymerization of diethyl aspartate using immobilized lipase CAL-B with up to 98% β-linkages and 63 repeating units on average in Solvent-free Lipase-catalyzed Synthesis: Unique Properties of Enantiopure D-and L-Polyaspartates and Their Complexation, Biomacromolecules 2016, 17, 362-370. However, the analytic data of Zhang et al. on the linkage of their polyaspartate are disputed in a subsequent publication by Gross et al., CAL-B Catalyzed Regioselective Bulk Polymerization of L-Aspartic Acid Diethyl Ester to α-Linked Polypeptides. It was demonstrated by Gross et al the polymerization of diethyl aspartate should be 94% α-linked polyaspartate with 50 repeating units on average using immobilized CAL-B under the same bulk conditions as reported by Zhang et al.

It is challenging to synthesize linear polylysine by chemoenzymatic bulk polymerization of lysine ester with the high regioselectivity of α-polylysine or ε-polylysine or synthesize linear polyarginine by chemoenzymatic bulk polymerization of arginine ester with the high regioselectivity of α-polyarginine.

### SUMMARY OF INVENTION

It is an object of the present invention to provide α- or ε-polylysine or α-polyarginine with desirable regioselectivity more efficiently.

It was found by the inventors that the object can be achieved by a process of preparation of linear polylysine or polyarginine through chemoenzymatic polymerization of lysine ester or arginine ester in bulk respectively.

Accordingly, in the first aspect, the present invention relates to a process for preparation of polylysine or polyarginine, which includes chemoenzymatic polymerization of a lysine ester in bulk in the presence of an immobilized protease to provide α-polylysine regioselectively or in the presence of an immobilized lipase to provide ε-polylysine regioselectively, or chemoenzymatic polymerization of an arginine ester in bulk in the presence of an immobilized protease to provide α-polylyarginine regioselectively.

In some embodiments according to the first aspect, the present invention relates to a process for preparation of polylysine, which includes
desalting a salt of lysine C₁₋₆-alkyl or aryl ester to provide a lysine C₁₋₆-alkyl or aryl ester, and
chemoenzymatic polymerization of the lysine C₁₋₆-alkyl or aryl ester in bulk in the presence of an immobilized protease to provide α-polylysine regioselectively or in the presence of an immobilized lipase to provide ε-polylysine regioselectively.

In some other embodiments according to the first aspect, the present invention relates to a process for preparation of polyarginine, which includes
desalting a salt of arginine C₁₋₆-alkyl or aryl ester to provide an arginine C₁₋₆-alkyl or aryl ester, and
chemoenzymatic polymerization of the arginine C₁₋₆-alkyl or aryl ester in bulk in the presence of an immobilized protease to provide α-polyarginine regioselectively.

In some preferable embodiments according to the first aspect, the present invention relates to a process for preparation of polylysine, which includes
desalting a mineral acid salt of lysine C₁₋₆-alkyl or aryl ester to provide a lysine C₁₋₆-alkyl or aryl ester, and
chemoenzymatic polymerization of the lysine C₁₋₆-alkyl or aryl ester in bulk in the presence of an immobilized protease selected from serine proteases such as trypsin and subtilisin or cysteine proteases such as papain to provide α-polylysine regioselectively.

In some preferable embodiments according to the first aspect, the present invention relates to a process for preparation of polylysine, which includes
desalting a mineral acid salt of lysine C₁₋₆-alkyl or aryl ester to provide a lysine C₁₋₆-alkyl or aryl ester, and
chemoenzymatic polymerization of the lysine C₁₋₆-alkyl or aryl ester in bulk in the presence of an immobilized lipase, preferably an immobilized lipase selected from the group consisting of Candida antarctica lipase B, Aspergillus niger lipase, Burkholderia cepacia (Pseudomonas cepacia) lipase and Rhizopus oryzae lipase to provide ε-polylysine regioselectively.

In some other preferable embodiments according to the first aspect, the present invention relates to a process for preparation of polyarginine, which includes
desalting a mineral acid salt of arginine C₁₋₆-alkyl or aryl ester to provide an arginine C₁₋₆-alkyl or aryl ester, and
chemoenzymatic polymerization of the arginine C₁₋₆-alkyl or aryl ester in bulk in the presence of an immobilized protease selected from serine proteases such as trypsin and subtilisin or cysteine proteases such as papain to provide α-polyarginine regioselectively.

In the second aspect, the present invention relates to a method for controlling regioselectivity of linear polylysine or linear polyarginine in the chemoenzymatic polymerization of a lysine ester or an arginine ester in bulk respectively, wherein an immobilized protease is used to provide α-polylysine or α-polyarginine regioselectively, or an immobilized lipase is used to provide ε-polylysine regioselectively.

In some embodiments according to the second aspect, the present invention relates to a method for controlling regioselectivity of linear polylysine or linear polyarginine in the chemoenzymatic polymerization of a lysine C₁₋₆-alkyl or aryl ester or an arginine C₁₋₆-alkyl or aryl ester in bulk respectively, wherein an immobilized protease selected from the group consisting of serine proteases such as trypsin and subtilisin and cysteine proteases such as papain is used to provide α-polylysine or α-polyarginine regioselectively.

In some other embodiments according to the second aspect, the present invention relates to a method for controlling regioselectivity of linear polylysine in the chemoenzymatic polymerization of a lysine C₁₋₆-alkyl or aryl ester in bulk, wherein an immobilized lipase, preferably an immobilized lipase selected from the group consisting of Candida antarctica lipase B, Aspergillus niger lipase, Aspergillus niger lipase, Burkholderia cepacia lipase and Rhizopus oryzae lipase is used to provide ε-polylysine regioselectively.

In the third aspect, the present invention relates to a process for preparation of polylysine or polyarginine, which includes chemoenzymatic polymerization of a lysine ester in bulk in the presence of a non-immobilized protease to provide α-polylysine regioselectively or in the presence of a non-immobilized lipase to provide ε-polylysine regioselectively, or chemoenzymatic polymerization of an arginine ester in bulk in the presence of a non-immobilized protease to provide α-prolylarginine regioselectively.

In the fourth aspect, the present invention relates to a method for controlling regioselectivity of linear polylysine or linear polyarginine in the chemoenzymatic polymerization of a lysine ester or an arginine ester in bulk respectively, wherein a non-immobilized protease is used to provide α-polylysine or α-polyarginine regioselectively, or a non-immobilized lipase is used to provide ε-polylysine regioselectively.

It has been surprisingly found by the inventors that polylysines with high α-regioselectivity or ε-regioselectivity and polyarginine with high α-regioselectivity may be provided by the process according to the first aspect or the method according to the second aspect. Particularly, polylysines with the ε-regioselectivity may be obtained, which is unknown in the art for the preparation of polylysine via chemoenzymatic polymerization.

### DETAILED DESCRIPTION

The singular forms “a”, “an” and “the” include plural referents unless the context clearly dictates otherwise. The terms “comprise(s)”, “comprising”, etc. are used interchangeably with “contain(s)”, “containing”, etc. and are to be interpreted in a non-limiting, open manner. That is, e.g., further components or elements can be present. The expressions “consist(s) of” or “consisting of” or cognates can be embraced within “comprise(s)” or “comprising” or cognates. The terms “include(s)”, “including”, etc. are to be interpreted in a non-limiting, open manner.

Herein, the term “regioselectivity” refers to a reaction that highly favors a single major product. In the present invention, the “regioselectivity” within the context of polylysine is intended to refer to a percentage of α-polylysine or ε-polylysine in the polylysines as formed in the chemoenzymatic polymerization of a lysine ester. The “regioselectivity” within the context of polyarginine is intended to refer to a percentage of α-polylysine in the polyarginines as formed in the chemoenzymatic polymerization of an arginine ester.

Herein, the phrase “chemoenzymatic polymerization of . . . in bulk” may also be referred to “chemoenzymatic bulk polymerization”.

Herein, the terms “lysine ester” and “arginine ester” refer to the respective free base forms of lysine ester and arginine ester.

In the first aspect, the present invention provides a process for preparation of polylysine or polyarginine, which includes chemoenzymatic polymerization of a lysine ester in bulk in the presence of an immobilized protease to provide α-polylysine regioselectively or in the presence an immobilized lipase to provide ε-polylysine regioselectively, or chemoenzymatic polymerization of an arginine ester in bulk in the presence of an immobilized protease to provide α-polyarginine regioselectively.

The present invention further provides a process for preparation of polylysine or polyarginine, which includes chemoenzymatic polymerization of a lysine ester in bulk in the presence of an immobilized protease or a non-immobilized protease to provide α-polylysine regioselectively or in the presence an immobilized lipase or a non-immobilized lipase to provide ε-polylysine regioselectively, or chemoenzymatic polymerization of an arginine ester in bulk in the presence of an immobilized protease or a non-immobilized protease to provide α-polyarginine regioselectively.

Suitable proteases for catalyzing peptide formation reaction with α-polylysine regioselectivity or α-polyarginine regioselectivity may include, but are not limited to, serine proteases (e.g., trypsin, α-chymotrypsin, elastase, carboxypeptidase, and subtilisin), cysteine proteases (e.g., papain, ficin, bromelain, analain, calpain, caspase, chymopapain, cathepsin, clostripain, and actinidin), metalloproteinases (e.g., thermolysin, collagenases, bacillolysin, dispase, vibriolysin, pseudolysin, stromelysin, and various bacterial derived neutral metalloproteases), aspartic proteases (e.g., pepsin, penicillopepsin, chymosin, cathepsin, renin, beta-secretase, plasmepsin, and retroviral proteases), liver esterase (e.g., pig liver esterase), alkaline protease, carbonic anhydrase, nonribosomal peptide synthetase, thrombin, cardosins A or B, or pronase.

In some embodiments, the protease for catalyzing peptide formation reaction with α-polylysine regioselectivity or α-polyarginine regioselectivity is selected from the group consisting of serine proteases such as trypsin, α-chymotrypsin, elastase, carboxypeptidase and subtilisin, and cysteine proteases such as papain, ficin, bromelain, analain, calpain, caspase, chymopapain, cathepsin, clostripain and actinidin. Particularly, the protease is selected from the group consisting of trypsin, subtilisin and papain.

The enzyme activity of protease, in particular trypsin, can be measured by using N α-benzoyl-DL-arginine-p-nitroanilide (BAPNA) assay as fully described hereinafter in the Example part. The trypsin used for the present invention has an activity of at least 0.05 U per g of immobilized trypsin, preferably at least 0.1 U/g, more preferably at least 0.3 U/g.

Suitable lipases for catalyzing peptide-formation reaction with ε-polylysine regioselectivity may include, but are not limited to, lipases (E.C. 3.1.1.3), for example lipase from Candida antarctica B, and lipase from Alcaligenes sp., Aspergillus sp., Mucor sp., Penicillium sp., Geotrichum sp., Rhizopus sp., Burkholderia sp., Candida sp., Pseudomonas sp., and Thermomyces sp.

In some embodiments, the lipase for catalyzing peptide-formation reaction with ε-polylysine regioselectivity is selected from the group consisting of lipase from Candida antarctica B (i.e., Candida antarctica lipase B (CALB)), lipase from Aspergillus niger (i.e., Aspergillus niger lipase), lipase from Burkholderia cepacia (i.e., Burkholderia cepacia lipase), and lipase from Rhizopus oryzae (Rhizopus oryzae lipase).

The enzyme activity of lipase can be measured by using amidation method as fully described hereinafter in the Example part. The unit of lipase activity can be expressed as u mol N-butylacetamide per minute per gram of immobilized lipase. Generally, the lipase used for the present invention has an activity of at least 5 μmol N-butylacetamide per minute per gram of immobilized lipase, particularly, at least 10 μmol N-butylacetamide per minute per gram of immobilized lipase.

In the process according to the present invention, the protease or lipase is used in an immobilized form in which the enzyme is carried on a carrier. There is no restriction to the carrier, which may be of any known organic or inorganic materials with suitable shapes or configurations.

In any process according to the present invention, the protease or lipase can be also used in a non-immobilized form in which the enzymes are not fixed or within solid carriers. According to any one embodiment of the present invention, the non-immobilized enzyme can be used as an alternative to the immobilized enzyme to achieve identical or similar technical effects.

Suitable carriers may include, but are not limited to, inorganic carriers selected from the group consisting of silica, activated charcoal, zeolite, alumina, titania, zirconia, silica magnesia, silica-zirconia-alumina, diatomaceous earth, bentonite, glass, ceramic and Kaolin, polymer carriers selected from the group consisting of polysaccharides (e.g., agarose, sepharose and cellulose), (meth)arylic polymers (e.g., homopolymers of (meth)acrylate, copolymers of (meth)acrylate, and polyacrylamide), polystyrene optionally cross-linked with divinylbenzene, epoxy resin, polyurethane, polypropylene, nylon, polyethylene glycol, collagen, starch, polyvinylchloride. The carrier may also be ion exchange resins.

For the purpose of the present invention, any useful commercially available immobilized protease and immobilized lipase may be used. Alternatively, the immobilized protease and the immobilized lipase may be prepared by suitable processes.

In some embodiments, the immobilized protease and the immobilized lipase may be prepared by a process comprising steps: a) preparing a liquid medium comprising a protease or a lipase in a buffer; b) introducing a carrier into the liquid medium, gentle stirring for a sufficient period and equilibrating without stirring for another sufficient period, and c) separating the resulted supernatant, and washing the obtained carrier carrying the protease or lipase. For example, the buffer in step a) may be a phosphate buffer saline. For example, the stirring in step b) may be carried out for at least 10 hours or at least 15 hours. The equilibrating in step b) may be carried out for at least 15 hours or at least 18 hours. The carrier may be pretreated by washing before step a). The obtained carrier carrying the protease or lipase from step c) may also be washed, for example with water, a sodium chloride solution or a buffer.

Suitable lysine esters may include but are not limited to alkyl or aryl esters of lysine (i.e., L-lysine or D-lysine). According to the present invention, L-lysine esters can be used for producing L-polylysine and D-lysine esters can be used for producing D-polylysine, in particular, L-lysine being used. The alkyl esters of lysine may for example be lysine C₁₋₆-alkyl esters, and the lysine aryl esters may for example be lysine phenyl ester or benzyl ester.

Suitable arginine esters may include, but are not limited to arginine alkyl or aryl esters, for example arginine C₁₋₆-alkyl esters, and arginine phenyl ester or benzyl ester.

In some embodiments, the lysine ester is selected from lysine C₁₋₆-alkyl esters, for example lysine C₁₋₄-alkyl esters, such as lysine methyl ester, lysine ethyl ester, lysine propyl ester and lysine butyl ester, among which lysine methyl ester and lysine ethyl ester are particularly useful for the present invention.

In some other embodiments, the arginine ester is selected from arginine C₁₋₆-alkyl esters, for example arginine C₁₋₄-alkyl esters, such as arginine methyl ester, arginine ethyl ester, arginine propyl ester and arginine butyl ester, among which arginine methyl ester and arginine ethyl ester are particularly useful for the present invention.

The lysine ester and the arginine ester may be prepared in accordance with any known processes or commercially available. Those esters may be commercially available in salt form. In that case, the process according to the present invention may include desalting a salt of lysine or arginine ester to provide the lysine or arginine ester in free base form. The salts of those esters are generally mineral acid salts, for example hydrochloric acid salts, hydrobromic acid salts, sulfuric acid salts, nitric acid salts or phosphoric acid salts, among which hydrochloric acid salts are particularly suitable for the present invention. Suitable salts of those esters may be salt of C₁₋₆-alkyl ester, particularly mineral acid salt of C₁₋₆-alkyl ester, more particularly hydrochloric acid salt of C₁₋₆-alkyl ester. Suitable salts of those esters may also include organic acid salts, for example formic acid salts, acetic acid salts, citric acid salts, lactic acid salts, fumaric acid salts, and propionic acids, etc.

The desalting may be carried out as a separate step before polymerization. For example, the desalting may be carried out in an aqueous medium with an alkaline pH for isolating an alkyl ester. Suitable pH may be at least 8.5, or at least 9.0, or at least 10. The alkaline pH may be provided by using suitable substances, for example sodium hydrogencarbonate or sodium hydroxide. Particularly, a saturated sodium hydrogencarbonate solution may be used as the aqueous medium with an alkaline pH. The desalting may be carried out at a temperature of 15° C. or lower, or 10° C. or lower, or 5° C. or lower, for example in an ice bath, a jacketed vessel with a circulating coolant or inside a cold room. The desalted product may be obtained by conventional means, for example by extracting the lysine or arginine ester free base from the aqueous solution to an organic phase. The treatment of the desalted product may also be carried out at a low temperature of 15° C. or lower, or 10° C. or lower, or 5° C. or lower.

The desalting may also be carried out by ion-exchanging with suitable anion exchange resin. The salt of lysine or arginine ester may be suspended in a suitable organic solvent such as chloroform or dichloromethane and subjected to the ion-exchanging at a temperature of for example 15° C. or lower, or 10° C. or lower. After ion-exchanging, the desalted product may be treated by conventional means.

Alternatively, an in-situ desalting may be employed in the process according to the present invention. The in-situ desalting may be carried out simply by incorporating an organic base, optionally together with a certain amount of water, before the chemoenzymatic bulk polymerization. Suitable organic bases may include, but are not limited to, any amines which will not adversely affect the chemoenzymatic bulk polymerization, for example tertiary amines (e.g., triethylamine N,N-Diisopropylethylamine), tertiary alkanolamines (e.g., triethanolamine), tertiary diamines (e.g., tetramethylethylenediamine), aromatic amines (e.g., pyridine).

The term “in-situ desalting” as used herein refers to desalting reaction carried out with the organic base which will be present in the bulk polymerization without removal.

In some specific embodiments, the chemoenzymatic bulk polymerization with in-situ desalting may be carried out. The bulk polymerization with in-situ desalting means the bulk polymerization is carried out with the presence of the organic base which is used in the desalting reaction without removal.

The chemoenzymatic bulk polymerization may be carried out at a temperature in the range of from −20° C. (minus 20° C.) to 80° C., preferably in the range of from 0° C. to 35° C. Generally, the chemoenzymatic bulk polymerization may be carried out for a period of at least 12 hours, particularly at least 20 hours. The chemoenzymatic bulk polymerization may be carried out for a period of up to 192 hours.

The polylysine or polyarginine as obtained from the chemoenzymatic bulk polymerization may be isolated by any conventional means. For example, the polylysine or polyarginine as obtained may be separated from the immobilized enzyme (i.e., protease or lipase) by means of washing with a solvent or dissolving into a solvent and then filtration. The solvent may be water, a suitable organic solvent or a combination thereof. Further recovery of the polylysine or polyarginine as obtained from the solvent may be carried out by any conventional means without restrictions.

It was found by the inventors that the immobilized enzyme, as obtained after the separation from the polylysine or polyarginine may be used for further bulk polymerization of a lysine or arginine ester in bulk respectively, to provide polylysine or polyarginine regioselectively. In other words, recycled immobilized enzyme may be used further for chemoenzymatic polymerization of a lysine or arginine ester to provide polylysine or polyarginine regioselectively.

Accordingly, in some embodiments, the process for preparation of polylysine or polyarginine according to the present invention may further include
separation of the immobilized enzyme and the polylysine or the polyarginine as obtained from the chemoenzymatic polymerization, and
applying the immobilized enzyme from the separation to a further chemoenzymatic polymerization of a lysine or arginine ester.

It will be understood that the polylysine or polyarginine as obtained will have a distribution of polylysines or polyarginine with various degrees of polymerization in a certain range. The process according to the present invention may provide a polylysine with a degree of polymerization (DP) range of from 2 to 60, particularly from 2 to 50. The degree of polymerization (DP) range of the polylysines as obtained by the process according to the present invention may have a DP at peak maximum in the range of from 4 to 25, particularly from 6 to 20.

In the embodiments wherein an immobilized protease is used to provide α-polylysine regioselectively, the DP range of the obtained polylysine may be from 2 to 60, particularly from 2 to 50 with a peak maximum of 4 to 15. The regioselectivity of α-polylysine may be at least 60%, preferably at least 70%, more preferably at least 80%.

In the embodiments wherein an immobilized lipase is used to provide ε-polylysine regioselectively, the DP range of the obtained polylysine may be from 2 to 50, particularly from 2 to 40 with a peak maximum of 4 to 10. The regioselectivity of ε-polylysine may be at least 70%, preferably at least 80%, more preferably at least 85%.

The process according to the present invention may provide polyarginines with a degree of polymerization (DP) range of from 2 to 30, particularly from 2 to 20. The degree of polymerization (DP) range of the polyarginines as obtained may have a DP at peak maximum in the range of from 2 to 5. The regioselectivity of α-polyarginine may be at least 75%, preferably at least 90%, more preferably at least 95%.

It has been surprisingly found by the inventors that polylysines with high α-regioselectivity or ε-regioselectivity as well as polyarginines with high α-regioselectivity are synthesized successfully by the chemoenzymatic bulk polymerization process in the presence of respective enzymes.

It has been surprisingly found by the inventors that polylysines with high α-regioselectivity or ε-regioselectivity as well as polyarginines with high α-regioselectivity are synthesized successfully by the chemoenzymatic bulk polymerization process in the presence of respective immobilized enzymes or non-immobilized enzymes.

Accordingly, in the second aspect, the present invention further provides a method for controlling regioselectivity of linear polylysine or polyarginine in the chemoenzymatic polymerization of a lysine ester or an arginine ester in bulk, wherein an immobilized protease is used to provide α-polylysine or α-polyarginine regioselectively, or an immobilized lipase is used to provide ε-polylysine regioselectively.

In the present method, the lysine ester may include, but is not limited to alkyl or aryl esters of lysine (i.e., L-lysine or D-lysine). The alkyl or aryl esters of lysine may for example be lysine C₁₋₆-alkyl esters, and the lysine aryl esters may for example be lysine phenyl ester or benzyl ester. The arginine esters may include, but is not limited to, arginine alkyl or aryl esters, for example arginine C₁₋₆-alkyl esters, and arginine phenyl ester or benzyl ester.

In some embodiments, the lysine ester is selected from lysine C₁₋₆-alkyl esters, for example lysine C₁₋₄-alkyl esters, for example lysine methyl ester, lysine ethyl ester, lysine propyl ester and lysine butyl ester, among which lysine methyl ester and lysine ethyl ester are particularly useful for the present invention.

In some other embodiments, the arginine ester is selected from arginine C₁₋₆-alkyl esters, for example arginine C₁₋₄-alkyl esters, such as arginine methyl ester, arginine ethyl ester, arginine propyl ester and arginine butyl ester, among which arginine methyl ester and arginine ethyl ester are particularly useful for the present invention.

Suitable proteases for catalyzing peptide formation reaction toward α-polylysine or α-polyarginine regioselectivity may include, but are not limited to, serine proteases (e.g., trypsin, α-chymotrypsin, elastase, carboxypeptidase, and subtilisin), cysteine proteases (e.g., papain, ficin, bromelain, analain, calpain, caspase, chymopapain, cathepsin, clostripain, and actinidin), metalloproteinases (e.g., thermolysin, collagenases, bacillolysin, dispase, vibriolysin, pseudolysin, stromelysin, and various bacterial derived neutral metalloproteases), aspartic proteases (e.g., pepsin, penicillopepsin, chymosin, cathepsin, renin, beta-secretase, plasmepsin, and retroviral proteases), liver esterase (e.g., pig liver esterase), alkaline protease, carbonic anhydrase, nonribosomal peptide synthetase, thrombin, cardosins A or B, or pronase.

In some embodiments, the protease for catalyzing peptide formation reaction toward α-polylysine or α-polyarginine regioselectivity is selected from the group consisting of serine proteases such as trypsin, α-chymotrypsin, elastase, carboxypeptidase and subtilisin, and cysteine proteases such as papain, ficin, bromelain, analain, calpain, caspase, chymopapain, cathepsin, clostripain and actinidin. Particularly, the protease is selected from the group consisting of trypsin, subtilisin and papain.

Suitable lipases for catalyzing peptide-formation reaction toward ε-polylysine regioselectivity may include, but are not limited to, lipases (E.C. 3.1.1.3), for example lipase from Candida antarctica B, and lipase from Alcaligenes sp., Aspergillus sp., Mucor sp., Penicillium sp., Geotrichum sp., Rhizopus sp., Burkholderia sp., Candida sp., Pseudomonas sp., and Thermomyces sp.

In some embodiments, the lipase for catalyzing peptide-formation reaction toward ε-polylysine regioselectivity is selected from the group consisting of lipase from Candida antarctica B (i.e., Candida antarctica lipase B (CALB)), lipase from Aspergillus niger (i.e., Aspergillus niger lipase), lipase from Burkholderia cepacia (i.e., Burkholderia cepacia lipase) and lipase from Rhizopus oryzae (Rhizopus oryzae lipase).

Suitable carriers for the immobilized protease and the immobilized lipase are those as described hereinabove within the context of the process for preparation of polylysine or polyarginine in the first aspect of the present invention.

In some embodiments according to the second aspect, the present invention relates to a method for controlling regioselectivity of linear polylysine or polyarginine in the chemoenzymatic polymerization of a lysine C₁₋₆-alkyl ester or an arginine C₁₋₆-alkyl ester in bulk respectively, wherein an immobilized protease selected from the group consisting of serine proteases such as trypsin and subtilisin and cysteine proteases such as papain is used to provide α-polylysine or α-polyarginine regioselectively.

In some embodiments according to the second aspect, the present invention relates to a method for controlling regioselectivity of linear polylysine or polyarginine in the chemoenzymatic polymerization of a lysine C₁₋₆-alkyl ester or an arginine C₁₋₆-alkyl ester in bulk respectively, wherein an immobilized protease or an non-immobilized protease selected from the group consisting of serine proteases such as trypsin and subtilisin and cysteine proteases such as papain is used to provide α-polylysine or α-polyarginine regioselectively.

In some other embodiments according to the second aspect, the present invention relates to a method for controlling regioselectivity of linear polylysine in the chemoenzymatic polymerization of a lysine C₁₋₆-alkyl ester in bulk, wherein an immobilized lipase selected from the group consisting of Candida antarctica lipase B, Aspergillus niger lipase, Burkholderia cepacia lipase and Rhizopus oryzae lipase is used to provide ε-polylysine regioselectively.

In some other embodiments according to the second aspect, the present invention relates to a method for controlling regioselectivity of linear polylysine in the chemoenzymatic polymerization of a lysine C₁₋₆-alkyl ester in bulk, wherein an immobilized lipase or a non-immobilized lipase selected from the group consisting of Candida antarctica lipase B, Aspergillus niger lipase, Burkholderia cepacia lipase and Rhizopus oryzae lipase is used to provide ε-polylysine regioselectively.

The chemoenzymatic bulk polymerization may be carried out at a temperature in the range of from −20° C. (minus 20° C.) to 80° C., preferably in the range of from 0° C. to 35° C. Generally, the chemoenzymatic bulk polymerization may be carried out for a period of at least 12 hours, particularly at least 20 hours. The chemoenzymatic bulk polymerization may be carried out for a period of up to 192 hours.

According to some embodiments of the present invention, the chemoenzymatic bulk polymerization may be carried out by adding additional polar organic solvent, wherein the polar organic solvent is selected from the group consisting of acetonitrile, dimethyl sulfoxide, acetone, methyl acetate, ethyl acetate, N,N-dimethylformamide, N,N-dimethylacetamide, tetrahydrofuran, propylene carbonate, dichlormethane, trichloromethane, sulfolane, hexamethylphosphoramide, 1,3-dimethyl-2-imidazolidinone, nitromethane, butanone, methanol, methyl formamide, ethanol, isopropanol, 1-propanol, tert-butyl alcohol, formamide, n-butanol, ethylene glycol, 2-methoxyethanol, 1-methoxy-2-propanol, glycerol, 1,2-propanediol, and 1,3-propanetriol, toluol.

The enzyme activity of protease, in particular trypsin, can be measured by using N α-benzoyl-DL-arginine-p-nitroanilide (BAPNA) assay as fully described hereinafter in the Example part. The trypsin used for the present invention has the activity of at least 0.05 U/g, preferably at least 0.1 U/g, more preferably at least 0.3 U/g.

In some embodiments of the present invention, the enzyme activity of the non-immobilized protease (native protease) is for example 10 U/mg to 50 U/mg, preferably 20 U/mg to 40 U/g. In some embodiments of the present invention, the enzyme activity of the non-immobilized protease is for example 500 USP/mg to 5000 USP/mg, for example 800 USP/mg to 4500 USP/mg.

The enzyme activity of lipase can be measured by using amidation method as fully described hereinafter in the Example part. The unit of lipase activity can be expressed as u mol N-butylacetamide per minute per gram of immobilized lipase. Generally, the lipase used for the present invention has the activity of at least 5 μmol N-butylacetamide per minute per gram of immobilized lipase, particularly, at least 10 μmol N-butylacetamide per minute per gram of immobilized lipase.

In the present method, the regioselectivity of α-polylysine may be at least 60%, preferably at least 70%, more preferably at least 80%. The regioselectivity of ε-polylysine may be at least 70%, preferably at least 80%, more preferably at least 85%. The regioselectivity of α-polyarginine may be at least 75%, preferably at least 90%, more preferably at least 95%.

### Embodiments

Various embodiments are listed below. It will be understood that the embodiments listed below may be combined with all aspects and other embodiments in accordance with the scope of the invention.
Embodiment 1: A process for preparation of polylysine or polyarginine, which includes chemoenzymatic polymerization of a lysine ester in bulk in the presence of an immobilized protease or a non-immobilized protease to provide α-polylysine regioselectively or in the presence an immobilized lipase or a non-immobilized lipase to provide ε-polylysine regioselectively, or chemoenzymatic polymerization of an arginine ester in bulk in the presence of an immobilized protease or a non-immobilized protease to provide α-polyarginine regioselectively.
Embodiment 2: The process according to Embodiment 1, wherein the lysine ester is selected from the group consisting of lysine alkyl esters and lysine aryl esters, and the arginine ester is selected from the group consisting of arginine alkyl esters and arginine aryl esters.
Embodiment 3: The process according to Embodiment 2, wherein the lysine ester is selected from lysine C₁₋₆-alkyl esters.
Embodiment 4: The process according to Embodiment 3, wherein the lysine ester is selected from lysine C₁₋₄-alkyl esters.
Embodiment 5: The process according to Embodiment 4, wherein the lysine ester is selected from lysine methyl ester, lysine ethyl ester, lysine propyl ester and lysine butyl ester.
Embodiment 6: The process according to Embodiment 2, wherein the lysine ester is selected from lysine phenyl ester and lysine benzyl ester.
Embodiment 7: The process according to Embodiment 2, wherein the arginine ester is selected from arginine C₁₋₆-alkyl esters.
Embodiment 8: The process according to Embodiment 7, wherein the arginine ester is selected from arginine C₁₋₄-alkyl esters.
Embodiment 9: The process according to Embodiment 8, wherein the arginine ester is selected from arginine methyl ester, arginine ethyl ester, arginine propyl ester and arginine butyl ester.
Embodiment 10: The process according to Embodiment 2, wherein the arginine ester is selected from arginine phenyl ester and arginine benzyl ester.
Embodiment 11: The process according to any of preceding Embodiments, wherein the protease is selected from the group consisting of serine proteases and cysteine proteases.
Embodiment 12: The process according to Embodiment 11, wherein the protease is selected from the group consisting of trypsin, α-chymotrypsin, elastase, carboxypeptidase and subtilisin.
Embodiment 13: The process according to Embodiment 11, wherein the protease is selected from the group consisting of papain, ficin, bromelain, analine, calpain, caspase, chymopapain, cathepsin, clostripain and actinidin.
Embodiment 14: The process according to Embodiment 11, wherein the protease is selected from the group consisting of trypsin, subtilisin and papain.
Embodiment 15: The process according to any of preceding Embodiments 1 to 10, wherein the lipase is selected from lipases (E.C. 3.1.1.3).
Embodiment 16: The process according to Embodiment 15, wherein the lipase is selected from the group consisting of lipase from Candida antarctica B (i.e., Candida antarctica lipase B (CALB)), lipase from Aspergillus niger (i.e., Aspergillus niger lipase), lipase from Burkholderia cepacia (i.e., Burkholderia cepacia lipase), and lipase from Rhizopus oryzae (Rhizopus oryzae lipase).
Embodiment 17: The process according to any of preceding Embodiments, which further includes desalting a salt of the lysine ester to provide the lysine ester, or desalting a salt of the arginine ester to provide the arginine ester.
Embodiment 18: The process according to Embodiment 17, wherein the salt of the lysine ester is a mineral acid salt of the lysine ester.
Embodiment 19: The process according to Embodiment 17, wherein the salt of the arginine ester is a mineral acid salt of the arginine ester.
Embodiment 20: The process according to any of preceding Embodiments, wherein the chemoenzymatic polymerization is carried out at a temperature in the range of from −20° C. (minus 20° C.) to 80° C.
Embodiment 21: The process according to Embodiment 20, wherein the chemoenzymatic polymerization is carried out at a temperature in the range of from 0° C. to 35° C.
Embodiment 22: The process according to any of preceding Embodiments, wherein the chemoenzymatic polymerization is carried out for a period of at least 12 hours, particularly at least 20 hours.
Embodiment 23: The process according to any of preceding Embodiments, wherein the chemoenzymatic polymerization is carried out for a period of up to 192 hours.
Embodiment 24: The process according to any of preceding Embodiments, which further includes separation of the immobilized enzyme or the non-immobilized enzyme from the polylysine or the polyarginine as obtained from the chemoenzymatic polymerization.
Embodiment 25: The process according to Embodiment 24, which further includes applying the immobilized enzyme or the non-immobilized enzyme from the separation to a further chemoenzymatic polymerization of a lysine ester or an arginine ester.
Embodiment 26: The process according to any of preceding Embodiments, wherein the polylysine has a degree of polymerization in the range from 2 to 60, and the polyarginine has a degree of polymerization in the range from 2 to 30.
Embodiment 27: The process according to Embodiment 26, wherein the polylysine has a degree of polymerization in the range from 2 to 50, and the polyarginine has a degree of polymerization in the range from 2 to 20.
Embodiment 28: The process according to Embodiment 26 or 27, wherein the range of the degree of polymerization of the polylysine has a peak maximum in the range of from 4 to 25, and the range of the degree of polymerization of the polyarginine has a peak maximum in the range of from 2 to 5.
Embodiment 29: The process according to Embodiment 28, wherein the range of the degree of polymerization of the polylysine has a peak maximum in the range of from 6 to 20.
Embodiment 30: A method for controlling regioselectivity of linear polylysine or polyarginine in the chemoenzymatic polymerization of a lysine ester or an arginine ester in bulk respectively, wherein an immobilized protease or a non-immobilized protease is used to provide α-polylysine or α-polyarginine regioselectively, or an immobilized lipase or a non-immobilized lipase is used to provide ε-polylysine regioselectively.
Embodiment 31: The method according to Embodiment 30, wherein the lysine ester is selected from the group consisting of lysine alkyl esters and lysine aryl esters, and the arginine ester is selected from the group consisting of arginine alkyl esters and arginine aryl esters.
Embodiment 32: The method according to Embodiment 31, wherein the lysine ester is selected from lysine C₁₋₆-alkyl esters.
Embodiment 33: The method according to Embodiment 32, wherein the lysine ester is selected from lysine C₁₋₄-alkyl esters.
Embodiment 34: The method according to Embodiment 33, wherein the lysine ester is selected from lysine methyl ester, lysine ethyl ester, lysine propyl ester and lysine butyl ester.
Embodiment 35: The method according to Embodiment 31, wherein the lysine ester is selected from lysine phenyl and lysine benzyl ester.
Embodiment 36: The method according to Embodiment 31, wherein the arginine ester is selected from arginine C₁₋₆-alkyl esters.
Embodiment 37: The method according to Embodiment 36, wherein the arginine ester is selected from arginine C₁₋₄-alkyl esters.
Embodiment 38: The method according to Embodiment 37, wherein the arginine ester is selected from arginine methyl ester, arginine ethyl ester, arginine propyl ester and arginine butyl ester.
Embodiment 39: The method according to Embodiment 31, wherein the arginine ester is selected from arginine phenyl ester and arginine benzyl ester.
Embodiment 40: The method according to any of Embodiments 30 to 39, wherein the protease is selected from the group consisting of serine proteases and cysteine proteases.
Embodiment 41: The method according to Embodiment 40, wherein the protease is selected from the group consisting of trypsin, α-chymotrypsin, elastase, carboxypeptidase and subtilisin.
Embodiment 42: The method according to Embodiment 40, wherein the protease is selected from the group consisting of papain, ficin, bromelain, analine, calpain, caspase, chymopapain, cathepsin, clostripain and actinidin.
Embodiment 43: The method according to Embodiment 40, wherein the protease is selected from the group consisting of trypsin, subtilisin and papain.
Embodiment 44: The method according to any of Embodiments 30 to 39, wherein the lipase is selected from lipases (E.C. 3.1.1.3).
Embodiment 45: The method according to Embodiment 44, wherein the lipase is selected from the group consisting of lipase from Candida antarctica B (i.e., Candida antarctica lipase B (CALB)), lipase from Aspergillus niger (i.e., Aspergillus niger lipase), lipase from Burkholderia cepacia (i.e., Burkholderia cepacia lipase) and lipase from Rhizopus oryzae (Rhizopus oryzae lipase).
Embodiment 46: The method according to any of embodiments 30 to 45, wherein the chemoenzymatic polymerization is carried out at a temperature in the range of from −20° C. (minus 20° C.) to 80° C., preferably in the range of from 0° C. to 35° C.
Embodiment 47: The method according to any of Embodiments 30 to 46, wherein the chemoenzymatic polymerization is carried out for a period of at least 12 hours, particularly at least 20 hours, preferably up to 192 hours.
Embodiment 48: The method according to any of Embodiments 30 to 47, wherein the regioselectivity of α-polylysine is at least 60%, preferably at least 70%.
Embodiment 49: The method according to any of Embodiments 30 to 47, wherein the regioselectivity of ε-polylysine may be at least 70%, preferably at least 80%.
Embodiment 50: The method according to any of Embodiments 30 to 47, wherein the regioselectivity of ε-polyarginine may be at least 75%, preferably at least 90%.

### EXAMPLES

Aspects of the present invention will be more fully illustrated by the following examples, which are set forth to illustrate certain aspects of the present invention and are not to be construed as limiting thereof.

### Enzyme Activity Measurement

The activities of the immobilized enzymes used in following Examples were measured in accordance with the respective procedures described below.

### Activity Measurement for Immobilized Lipase

The activity of immobilized lipases for the chemoenzymatic bulk polymerization of lysine ester was estimated by using their activity in the reaction of n-butylamine with benzyl acetate to form n-butyl acetamide as shown below.

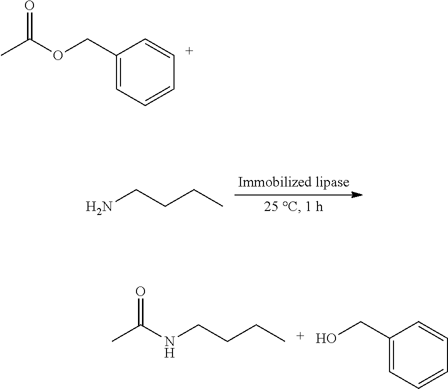

50 μL of benzyl acetate and 50 μL of n-butylamine were subjected to a reaction in the presence of 20 mg of immobilized lipase at 25° C. for 60 min, and then the conversion was determined by ¹H NMR (without additional solvent). The amount of N-butylacetamide produced per minute per gram of the immobilized lipase, expressed as μmol N-butylacetamide/min/g, was reported as the activity of the immobilized lipases.

| | Activity |
|---|---|
| Lipase | (μmol N-butylacetamide/min/g) |
| Immobilized CALB purchased from | 52.44 |
| Sigma-Aldrich | |
| Immobilized CALB purchased from | 46.61 |
| Novozymes (as used in Example 11) | |
| Immobilized CALB purchased from | 40.79 |
| Novozymes (as used in Example 13) | |
| Amano Lipase CL IM purchased | 29.13 |
| from Amano | |
| Amano lipase PS (Sigma) | 17.48 |

### Activity Measurement for Immobilized Trypsin

The activity of the immobilized trypsin was estimated using Na-benzoyl-DL-arginine-p-nitroanilide (BAPNA) assay. An absorbance at 410 nm was used to measure the activity of enzyme. One Unit (U) of enzyme activity was defined as the amount of enzyme capable of hydrolyzing 1 μmol BAPNA per min at pH 8.2, at 25° C., in the presence of Ca²⁺. The amount of BAPNA hydrolyzed per minute per gram of immobilized trypsin, expressed as U/g, was reported as the activity of the immobilized trypsin.

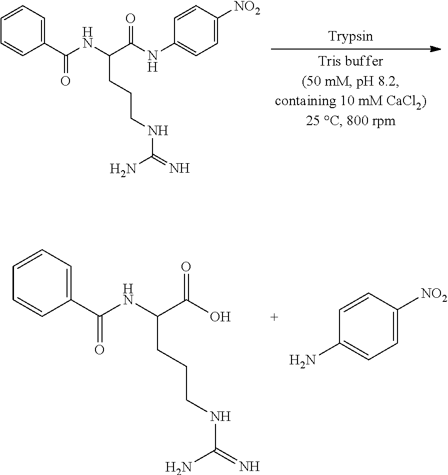 [SMILES: N=C(N)NCCCC(NC(=O)c1ccccc1)C(=O)Nc1ccc([N+](=O)[O-])cc1 ; N=C(N)NCCCC(NC(=O)c1ccccc1)C(=O)O ; "+" ; Nc1ccc([N+](=O)[O-])cc1]

The activity of the commercial immobilized trypsin purchased from Thermo Fisher Scientific is 0.812 U/g. The activity of the immobilized trypsin as prepared is provided in corresponding Examples.

### Example 1

### Preparation of Lysine Ethyl Ester (Lys-OEt) by Desalting Lys-OEt Dihydrochloride

5 g of lysine ethyl ester dihydrochloride (Lys-OEt dihydrochloride) was dissolved in a saturated sodium hydrogencarbonate solution in an ice bath. Then, the pH was adjusted to 10.5 by addition of a 5 M sodium hydroxide (NaOH) solution. The resultant aqueous solution was poured into a 300 mL extraction funnel to extract the Lys-OEt from the solution with an excess amount of chloroform by shaking, followed by separation of phases. All materials were cooled beforehand, to suppress the thermal condensation of the Lys-OEt during extraction, while the whole process was conducted in a cold room. Finally, chloroform was evaporated at 10° C. using vacuum pump, yielding Lys-OEt in liquid state.

### Bulk Polymerization of Lys-OEt

200 μL of Lys-OEt was poured into a polypropylene microtube, and 10 mg of acrylic resin with immobilized CALB purchased from Sigma-Aldrich was added. The bulk polymerization was performed at 40° C. for 24 h. Afterwards, the mixture was cooled to −30° C. to stop the reaction. The crude product after filtration was characterized by MALDI coupled to time-of-flight mass spectrometry (MALDI-TOF MS) and ¹H NMR, to determine the degree of polymerization (DP) range, monomer conversion and regioselectivity. The DP range was 2 to 11 with 4 at peak maximum, the conversion was 37% and regioselectivity (ε) was 72%.

### Example 2

The process was carried out according to Example 1, with the exception that 20 mg of acrylic resin with immobilized CALB purchased from Sigma-Aldrich was used for the bulk polymerization. The DP range was 2 to 12 with 5 at peak maximum, the conversion was 42% and the regioselectivity (ε) was 77%.

### Example 3

The process was carried out according to Example 1, with the exception that 30 mg of acrylic resin with immobilized CALB purchased from Sigma-Aldrich was used for the polymerization. The DP range was 2 to 13 with 5 at peak maximum, the conversion was 47% and the regioselectivity (ε) was 85%.

### Example 4

The process was carried out according to Example 3, with the exception that the temperature was maintained at 30° C. during the bulk polymerization. The DP range was 2 to 12 with 4 at peak maximum, the conversion was 32% and the regioselectivity (ε) was 88%.

### Example 5

The process was carried out according to Example 3, with the exception that the temperature was maintained at 20° C. during the bulk polymerization. The DP range was 2 to 16 with 6 at peak maximum, the conversion was 61% and the regioselectivity (ε) was 92%.

### Example 6

The process was carried out according to Example 3, with the exception that the temperature was maintained at 10° C. during the bulk polymerization. The DP range was 2 to 12 with 4 at peak maximum, the conversion was 56% and the regioselectivity (ε) was 96%.

### Example 7

200 μL of Lys-OEt as prepared according to Example 1 was poured into a polypropylene microtube, and 10 mg of Amano lipase PS (lipase immobilized on diatomaceous earth, purchased from Sigma-Aldrich) was added. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 2 to 12 with 4 at peak maximum, the conversion was 54% and the regioselectivity (ε) was 73%.

### Example 8

### Preparation of Lysine Ethyl Ester (Lys-OEt) by Desalting Lys-OEt Dihydrochloride

5 g of Lys-OEt dihydrochloride was suspended in 50 ml chloroform under vigorous stirring. Then, 20 g of strong anion exchange resin Amberlite IRN78, purchased from Sigma Aldrich, was added and equilibrated under mild stirring for 30 min at 10° C. All materials were cooled beforehand to suppress the thermal condensation of the Lys-OEt during extraction, while the whole process was conducted in a cold room. Finally, the exchange resin was filtered off and chloroform was evaporated at 10° C. using vacuum pump, yielding Lys-OEt in liquid state.

### Bulk Polymerization of Lys-OEt

200 μL of Lys-OEt was poured into a polypropylene microtube, and 30 mg of acrylic resin with immobilized CALB purchased from Sigma-Aldrich was added. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 3 to 22 with 6 at peak maximum, the conversion was 64% and the regioselectivity (E) was 99%.

### Example 9

The process was carried out according to Example 6, with the exception that the bulk polymerization was performed for 96 h. The DP range was 2 to 15 with 5 at peak maximum, the conversion was 68% and the regioselectivity (ε) was 99%.

### Example 10

### Preparation of Lysine Ethyl Ester (Lys-OEt) by Desalting Lys-OEt Dihydrochloride

30 g of Lys-OEt dihydrochloride was suspended in 300 ml chloroform under vigorous stirring. Then, 120 g of strong anion exchange resin Amberlite IRN78, purchased from Sigma Aldrich, was added and equilibrated under mild stirring for 60 min at 4° C. All materials were cooled beforehand, to suppress the thermal condensation of the Lys-OEt during extraction, while the whole process was conducted in a cold room. The resin was filtered off and the solution was dried using anhydrous NaSO₄. Finally, chloroform was evaporated at 10° C. using vacuum pump, yielding Lys-OEt in liquid state.

### Bulk Polymerization of Lys-OEt

The Lys-OEt was polymerized in bulk according to Example 8. The DP range was 2 to 19 with 6 at peak maximum, the conversion was 100% and the regioselectivity (ε) was 96%.

### Example 11

200 μL of Lys-OEt as prepared according to Example 1 was poured into a polypropylene microtube, and 30 mg of Novozym 435 (CALB immobilized on polymeric resin, purchased from Novozymes) was added. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 2 to 16 with 5 at peak maximum, the conversion was 100% and the regioselectivity (E) was 100%.

### Example 12

The process was carried out according to Example 11, with the exception that 60 mg of Novozym 435 (CALB immobilized on polymeric resin, purchased from Novozymes) was used for the polymerization. The DP range was 3 to 23 with 6 at peak maximum, the conversion was 100% and the regioselectivity (ε) was 100%.

### Example 13

200 μL of Lys-OEt as prepared according to Example 1 was poured into a polypropylene microtube and 30 mg of Amano lipase CL IM (lipase immobilized on polyacrylate resin (Available from Amano Enzyme)), was added. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 3 to 28 with 5 at peak maximum, the conversion was 100% and the regioselectivity (ε) was 100%.

### Example 14

200 μL of Lys-OEt as prepared according to Example 1, was poured into a polypropylene microtube. Subsequently, 10 mg of agarose beads with immobilized trypsin (purchased from Thermo Fisher Scientific) that had been previously washed five times with water was added to the microtube. The bulk polymerization was performed at 40° C. for 24 h. Afterwards, the mixture was cooled to −30° C. to stop the reaction. The crude product after filtration was characterized by MALDI-TOF MS and ¹H NMR. The DP range was 2 to 8 with 4 at peak maximum, the conversion was 79% and the regioselectivity (α) was 64%.

### Example 15

The process was carried out according to Example 14, with the exception that 30 mg of agarose beads with immobilized trypsin purchased from Thermo Fisher Scientific was used for the polymerization. The DP range was 2 to 8 with 4 at peak maximum, the conversion was 63% and the regioselectivity (α) was 75%.

### Example 16

200 μL of Lys-OEt as prepared according to Example 1 was poured into a polypropylene microtube. Subsequently, 30 mg of agarose beads with immobilized trypsin (purchased from Thermo Fisher Scientific) that had been previously washed five times with water was added to the microtube. The bulk polymerization was performed at 20° C. for 72 h. Afterwards, the mixture was cooled to −30° C. to stop the reaction. The crude product after filtration was characterized by MALDI-TOF MS and ¹H NMR. The DP range was 3 to 22 with 8 at peak maximum, the conversion was 69% and the regioselectivity (α) was 64%.

### Example 17

200 μL of Lys-OEt as prepared according to Example 1 was poured into a polypropylene microtube. Subsequently, 30 mg of agarose beads with immobilized trypsin (purchased from Thermo Fisher Scientific) that had been previously washed five times with water was added to the microtube. The bulk polymerization was performed at 10° C. for 168 h, while crude products were taken at 48 h, 72 h and 168 h. The final mixture was cooled to −30° C. to stop the reaction. The crude products were characterized by MALDI-TOF MS and ¹H NMR. The results are summarized in Table 1.

TABLE 1

| Time | DP range | DP_{peak max} | Conversion | Regioselectivity (α) |
|---|---|---|---|---|
| 48 h | 2 to 14 | 5 | 50% | 61% |
| 72 h | 2 to 14 | 5 | 57% | 72% |
| 168 h | 2 to 20 | 7 | 65% | 69% |

### Example 18

200 μL of Lys-OEt as prepared according to Example 1, was poured into a polypropylene microtube. Subsequently, 60 mg of agarose beads with immobilized trypsin (purchased from Thermo Fisher Scientific) that had been previously washed five times with water was added to the microtube. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 4 to 27 with 10 at peak maximum, the conversion was 57% and the regioselectivity (α) was 87%.

### Example 19

The process was carried out according to Example 18, with the exception that 90 mg of agarose beads with immobilized trypsin purchased from Thermo Fisher Scientific was used for the polymerization. The DP range was 5 to 30 with 11 at peak maximum, the conversion was 45% and the regioselectivity (α) was 90%.

### Example 20

200 μL of Lys-OEt as prepared according to Example 1 was poured into a polypropylene microtube. Subsequently, 120 mg of agarose beads with immobilized trypsin (purchased from Thermo Fisher Scientific) that had been previously washed five times with water, was added to the microtube. The bulk polymerization was performed at 30° C. for 72 h. Afterwards, the mixture was cooled to −30° C. to stop the reaction. The crude product after filtration was characterized by MALDI-TOF MS and ¹H NMR. The DP range was 2 to 26 with 5 at peak maximum, the conversion was 89% and the regioselectivity (α) was 82%.

### Example 21

The process was carried out according to Example 20, with the exception that the temperature was maintained at 20° C. during the bulk polymerization. The DP range was 2 to 27 with 8 at peak maximum, the conversion was 90% and the regioselectivity (α) was 86%.

### Example 22

200 μL of Lys-OEt as prepared according to Example 1 was poured into a polypropylene microtube. Subsequently, 120 mg of agarose beads with immobilized trypsin (purchased from Thermo Fisher Scientific) that had been previously washed five times with water was added to the microtube. The bulk polymerization was performed at 10° C. for 96 h, while crude products were taken at 24 h, 48 h and 72 h. The final mixture was cooled to −30° C. to stop the reaction. The crude products were characterized by MALDI-TOF MS and ¹H NMR. The results are summarized in Table 2.

TABLE 2

| Time | DP range | DP_{peak max} | Conversion | Regioselectivity (α) |
|---|---|---|---|---|
| 24 h | 2 to 28 | 8 | 90 | 92 |
| 48 h | 2 to 34 | 10 | 91 | 90 |
| 72 h | 2 to 39 | 11 | 91 | 90 |
| 96 h | 2 to 39 | 19 | 93 | 90 |

### Example 23

The process was carried out according to Example 20, with the exception that the temperature was maintained at 4° C. during the bulk polymerization. The DP range was 2 to 33 with 9 at peak maximum, the conversion was 87% and the regioselectivity (α) was 95%.

### Example 24

### Recycling Immobilized Trypsin

The recyclability of the immobilized trypsin was tested by conducting the bulk polymerization according to Example 22 at 10° C. for 72 h. Following the initial polymerization, the product was washed with 1000 μL of water, centrifuged at 6000 rpm for 1 min and the supernatant containing the resultant polylysine was removed. This washing step was repeated five times. Afterwards, 200 μL of Lys-OEt was added to initiate the next polymerization cycle. Six repeated polymerization cycles were conducted. The polymer from each cycle was characterized. The results are summarized in Table 3.

TABLE 3

| Recycle | DP range | DP_{peak max} | Conversion | Regioselectivity (α) |
|---|---|---|---|---|
| 0 | 2 to 38 | 9 | 93 | 90 |
| 1 | 2 to 38 | 10 | 89 | 88 |
| 2 | 2 to 38 | 10 | 87 | 89 |
| 3 | 2 to 33 | 9 | 91 | 87 |
| 4 | 2 to 33 | 8 | 79 | 88 |
| 5 | 2 to 33 | 7 | 72 | 80 |
| 6 | 2 to 36 | 8 | 76 | 80 |

### Example 25

### Preparation of Lysine Ethyl Ester (Lys-OEt) by Desalting Lys-OEt Dihydrochloride

30 g of Lys-OEt dihydrochloride was suspended in 300 ml chloroform under vigorous stirring. Then, 120 g of strong anion exchange resin Amberlite IRN78 purchased from Sigma Aldrich was added and equilibrated under mild stirring for 60 min at 4° C. All materials were cooled beforehand, to suppress the thermal condensation of the Lys-OEt during extraction, while the whole process was conducted in a cold room. Finally, the exchange resin was filtered off and chloroform was evaporated at 10° C. using vacuum pump, yielding Lys-OEt in liquid state.

### Bulk Polymerization of Lys-OEt

The Lys-OEt was polymerized in bulk according to Example 22, with the exception that the polymerization was performed for 72 h. The DP range was 3 to 31 with 10 at peak maximum, the conversion was 81% and the regioselectivity (α) was 89%.

### Example 26

### Immobilization of Trypsin

500 mg of polymeric resin ECR8209F (Purolite) was washed with water at a resin:water ratio of 1:2 w/v and the excess liquid was filtered out. The washing was repeated 4 times. The washed resin was added to a 25 mg/ml solution of trypsin (1:250 from porcine pancreas, for cell culture, Wako) in 1× phosphate buffer saline pH 7.4, with a resin:buffer ratio of 1:4 w/v. The resultant slurry was gently mixed for 18 h using a tube mixer and was left to equilibrate without mixing for another 20 h. Subsequently, the supernatant was collected, and the resin was washed with water four times under gentle mixing. The enzyme immobilization yield was 71%, estimated based on the trypsin concentration remaining in the supernatant, using bicinchoninic acid assay (BCA assay). The activity of the immobilized trypsin was 1.16 U/g, estimated using Na-benzoyl-DL-arginine-p-nitroanilide (BAPNA) assay.

### Bulk Polymerization of Lys-OEt

200 μL of Lys-OEt as prepared according to Example 25 was poured into a polypropylene microtube. Subsequently, 84 mg of the resin ECR8209F with trypsin immobilized according to the above procedure was added to the microtube. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 3 to 46 with 10 at peak maximum, the conversion was 87% and the regioselectivity (α) was 85%.

### Example 27

### Immobilization of Trypsin

Trypsin was immobilized on polymeric resin ECR8209F (Purolite) according to the procedure in Example 26, with the exception that the concentration of trypsin in the immobilization buffer was 12.5 mg/ml. The enzyme immobilization yield was 69%, and the enzyme activity was 0.696 U/g.

### Bulk Polymerization of Lys-OEt

200 μL of Lys-OEt as prepared according to Example 25 was poured into a polypropylene microtube. Subsequently, 140 mg of the resin ECR8209F with trypsin immobilized according to the above procedure was added to the microtube. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 3 to 46 with 11 at peak maximum, the conversion was 96% and the regioselectivity (α) was 92%.

### Example 28

### Immobilization of Trypsin

Trypsin was immobilized on polymeric resin ECR8285 (Purolite) according to the procedure in Example 27, with the exception that polymeric resin ECR8285 was used. The enzyme immobilization yield was 86%, and the enzyme activity was 0.000968 U/g.

### Bulk Polymerization of Lys-OEt

200 μL of Lys-OEt as prepared according to Example 25 was poured into a polypropylene microtube. Subsequently, 420 mg of the resin ECR8285 with trypsin immobilized according to the above procedure was added to the microtube. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 3 to 42 with 9 at peak maximum, the conversion was 83% and the regioselectivity (α) was 84%.

### Example 29

### Immobilization of Trypsin

Trypsin was immobilized on polymeric resin Chromalite MEP/M (Purolite) according to the procedure in Example 27, with the exception that resin Chromalite MEP/M was used. The enzyme immobilization yield was 40%, and the enzyme activity was 0.348 U/g.

### Bulk Polymerization of Lys-OEt

200 μL of Lys-OEt as prepared according to Example 25 was poured into a polypropylene microtube. Subsequently, 280 mg of the resin Chromalite MEP/M with trypsin immobilized according to the above procedure was added to the microtube. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 3 to 41 with 10 at peak maximum, the conversion was 85% and the regioselectivity (α) was 88%.

### Example 30

### Immobilization of Trypsin

Trypsin was immobilized on polymeric resin ECR8209F (Purolite) according to the procedure in Example 26, with the exception that 2× phosphate buffer saline pH 7.4 was used for dissolving trypsin. The enzyme immobilization yield was 67%, and the enzyme activity was 0.784 U/g.

### Bulk Polymerization of Lys-OEt

200 μL of Lys-OEt as prepared according to Example 25 was poured into a polypropylene microtube. Subsequently, 79 mg of the resin ECR8209F with trypsin immobilized according to the above procedure was added to the microtube. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 3 to 39 with 10 at peak maximum, the conversion was 90% and the regioselectivity (α) was 92%.

### Example 31

### Immobilization of Trypsin

Trypsin was immobilized on polymeric resin ECR8209F (Purolite) according to the procedure in Example 26, with the exception that the concentration of trypsin in the immobilization buffer was 62.5 mg/ml. The enzyme immobilization yield was 53%, and the enzyme activity was 0.232 U/g.

### Bulk Polymerization of Lys-OEt

200 μL of Lys-OEt as prepared according to Example 25 was poured into a polypropylene microtube. Subsequently, 255 mg of the resin ECR8209F with trypsin immobilized according to the above procedure was added to the microtube. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 3 to 33 with 7 at peak maximum, the conversion was 97% and the regioselectivity (α) was 88%.

### Example 32

### Immobilization of Trypsin

Trypsin was immobilized on polymeric resin ECR8209F (Purolite) according to the procedure in Example 26, with the exception that 10 g of the resin ECR8209F were used. The enzyme immobilization yield was 49%, and the enzyme activity was 0.420 U/g.

### Bulk Polymerization of Lys-OEt

200 μL of Lys-OEt as prepared according to Example 25 was poured into a polypropylene microtube. Subsequently, 88 mg of the resin ECR8209F with trypsin immobilized according to the above procedure was added to the microtube. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 3 to 37 with 11 at peak maximum, the conversion was 95% and the regioselectivity (α) was 85%.

### Example 33

### Immobilization of Trypsin

Trypsin was immobilized on polymeric resin ECR8209F (Purolite) according to the procedure in Example 26, with the exception that 0.5 M NaCl aqueous solution was used for resin washing after enzyme immobilization. The enzyme activity was 0.665 U/g.

### Bulk Polymerization of Lys-OEt

200 μL of Lys-OEt as prepared according to Example 25 was poured into a polypropylene microtube. Subsequently, 111 mg of the resin ECR8209F with trypsin immobilized according to the above procedure was added to the microtube. The bulk polymerization was performed at 10° C. for 72 h. Afterwards, the mixture was cooled to −30° C. to stop the reaction. The crude product after filtration was characterized by MALDI-TOF MS and ¹H NMR. The DP range was 3 to 42 with 11 at peak maximum, the conversion was 88% and the regioselectivity (α) was 81%.

### Example 34

Bulk Polymerization of Lys-OEt with In-Situ Desalting

200 mg of Lys-OEt dihydrochloride was thoroughly mixed in a polypropylene microtube with 150 μL of triethanolamine at 10° C. Subsequently, 84 mg of agarose beads with immobilized trypsin (purchased from Thermo Fisher Scientific) that had been previously washed five times with water was added to the tube, to start the chemoenzymatic polymerization. The bulk polymerization proceeded at 10° C. for 72 h. Afterwards, the mixture was cooled to −30° C. to stop the reaction. The crude product after filtration was characterized by MALDI-TOF MS and ¹H NMR. The DP range was 2 to 21 with 5 at peak maximum, the conversion was 94% and regioselectivity (α) was 88%.

### Example 35

Bulk Polymerization of Lys-OEt with In-Situ Desalting

200 mg of Lys-OEt dihydrochloride was thoroughly mixed in a polypropylene microtube with 100 μL of triethanolamine and 50 μL of water at 10° C. Subsequently, 84 mg of agarose beads with immobilized trypsin (purchased from Thermo Fisher Scientific) that had been previously washed five times with water was added to the tube, to start the chemoenzymatic polymerization. The bulk polymerization proceeded at 10° C. for 72 h. Afterwards, the mixture was cooled to −30° C. to stop the reaction. The crude product after filtration was characterized by MALDI-TOF MS and 1H NMR. The DP range was 2 to 26 with 7 at peak maximum, the conversion was 81% and the regioselectivity (α) was 93%.

### Example 36

Bulk Polymerization of Lys-OEt with In-Situ Desalting

Lys-OEt was desalted and then polymerized in bulk, according to Example 35, with the exception that 100 μL of triethylamine was used as the organic base for desalting. The DP range was 2 to 11 with 4 at peak maximum, the conversion was 63% and the regioselectivity (α) was 91%.

### Example 37

Bulk Polymerization of Lys-OEt with In-Situ Desalting

Lys-OEt was desalted and then polymerized in bulk according to Example 35, with the exception that 100 μL of tetramethylethylenediamine was used as the organic base for desalting. The DP range was 2 to 26 with 4 at peak maximum, the conversion was 44% and the regioselectivity (α) was 100%.

### Example 38

Bulk Polymerization of Arg-OEt with In-Situ Desalting

200 mg of arginine ethyl ester (Arg-OEt) dihydrochloride was thoroughly mixed in a polypropylene microtube with 100 μL of triethanolamine at 10° C. Subsequently, 84 mg of agarose beads with immobilized trypsin (purchased from Thermo Fisher Scientific) that had been previously washed five times with water was added to the tube, to start the chemoenzymatic polymerization. The bulk polymerization proceeded at 10° C. for 168 h, while crude products were taken at 24 h, 72 h and 168 h. The final mixture was cooled to −30° C. to stop the reaction. The crude products after filtration were characterized by MALDI-TOF MS and ¹H NMR. The results are summarized in Table 4.

TABLE 4

| Time | DP range | DP_{peak max} | Conversion | Regioselectivity (α) |
|---|---|---|---|---|
| 24 h | 2 to 9 | 3 | 24 | 100 |
| 72 h | 2 to 11 | 3 | 40 | 100 |
| 168 h | 2 to 8 | 3 | 18 | 100 |

### Example 39

Bulk Polymerization of Arg-OEt with In-Situ Desalting

Lys-OEt was desalted and then polymerized in bulk according to Example 38, with the exception that the bulk polymerization was conducted at 30° C. for 72 h. The DP range was 2 to 7 with 3 at peak maximum, the conversion was 29% and the regioselectivity (α) was 100%.

### Example 40

Bulk Polymerization of Arg-OEt with In-Situ Desalting

Arg-OEt was desalted and then polymerized in bulk according to Example 38, with the exception that the bulk polymerization was conducted at 60° C. for 72 h. The DP was 2 to 7 with 2 at peak maximum, the conversion was 39% and the regioselectivity (α) was 100%.

### Example 41

Bulk Polymerization of Arg-OEt with In-Situ Desalting

200 mg of arginine ethyl ester (Arg-OEt) dihydrochloride was thoroughly mixed in a polypropylene microtube with 100 μL of triethylamine at 10° C. Subsequently, 84 mg of agarose beads with immobilized trypsin (purchased from Thermo Fisher Scientific) that had been previously washed five times with water was added to the tube, to start the chemoenzymatic polymerization. The bulk polymerization proceeded at 10° C. for 72 h. The DP range was 2 to 8 with 3 at peak maximum, the conversion was 56% and the regioselectivity (α) was 100%.

Although the invention herein has been described with reference to particular embodiments, it is to be understood that these embodiments are merely illustrative of the principles and applications of the present invention. It will be apparent to those of skill in the art that various modifications and variations can be made to the method and apparatus of the present invention without departing from the spirit and scope of the invention. Thus, it is intended that the present invention include modifications and variations that are within the scope of the appended claims and their equivalents.

### Example 42

Lys-OEt was desalted and then polymerized in bulk, according to Example 10. The bulk polymerization was performed at 10° C. for 24 h, while crude samples were taken between 1 h and 24 h. The results from characterization of the crude samples are available in Table 5.

TABLE 5
Parameters from process in Example 42.

| | | | | Regioselectivity |
|---|---|---|---|---|
| Time | DP range | DPpeak max | Conversion | (ε) |
| 1 h | 2-8 | 3 | 28 | 100 |
| 6 h | 2-10 | 3 | 84 | 98 |
| 12 h | 2-13 | 4 | 91 | 100 |
| 24 h | 2-16 | 5 | 100 | 99 |

### Example 43

The process was carried out according to Example 42 for 72 h, with the exception that 5 μl of acetone were added in the microtube for the polymerization. The DP range was 3 to 29 with 6 at peak maximum, the conversion was 100% and the regioselectivity (ε) was 95%.

### Example 44

The process was carried out according to Example 43, with the exception that 20 μl of acetone were added in the microtube for the polymerization. The DP range was 3 to 23 with 6 at peak maximum, the conversion was 100% and the regioselectivity (E) was 98%.

### Example 45

The process was carried out according to Example 43, with the exception that 5 μl of acetonitrile were added in the microtube for the polymerization. The DP range was 4 to 30 with 6 at peak maximum, the conversion was 100% and the regioselectivity (ε) was 99%.

### Example 46

The process was carried out according to Example 43, with the exception that 10 μl of acetonitrile were added in the microtube for the polymerization. The DP range was 4 to 30 with 6 at peak maximum, the conversion was 100% and the regioselectivity (8) was 97%.

### Example 47

The process was carried out according to Example 43, with the exception that 20 μl of acetonitrile were added in the microtube for the polymerization. The DP range was 3 to 32 with 5 at peak maximum, the conversion was 100% and the regioselectivity (ε) was 100%.

### Example 48

The process was carried out according to Example 43, with the exception that 5 μl of ethanol were added in the microtube for the polymerization. The DP range was 3 to 18 with 5 at peak maximum, the conversion was 100% and the regioselectivity (ε) was 93%.

### Example 49

The process was carried out according to Example 43, with the exception that 10 μl of ethanol were added in the microtube for the polymerization. The DP range was 3 to 23 with 6 at peak maximum, the conversion was 100% and the regioselectivity (ε) was 93%.

### Example 50

The process was carried out according to Example 43, with the exception that 20 μl of ethanol were added in the microtube for the polymerization. The DP range was 3 to 18 with 6 at peak maximum, the conversion was 100% and the regioselectivity (ε) was 91%.

### Example 51

The process was carried out according to Example 43, with the exception that 50 μl of ethanol were added in the microtube for the polymerization. The DP range was 3 to 20 with 6 at peak maximum, the conversion was 100% and the regioselectivity (ε) was 89%.

### Example 52

The process was carried out according to Example 43, with the exception that 5 μl of glycerol were added in the microtube for the polymerization. The DP range was 4 to 31 with 6 at peak maximum, the conversion was 50% and the regioselectivity (ε) was 94%.

### Example 53

The process was carried out according to Example 43, with the exception that 10 μl of glycerol were added in the microtube for the polymerization. The DP range was 2 to 17 with 5 at peak maximum, the conversion was 40% and the regioselectivity (ε) was 99%.

### Example 54

### Demineralization of Lys-OEt Dihydrochloride:

100 g of anion exchange resin type II Amberlite IRA410 (Cl form) purchased from Sigma Aldrich were washed with 200 ml of 1 M NaOH aqueous solution, in order to be converted to the OH form. The resin was further washed with 200 ml of deionized water, followed by 50 ml of ethanol. Then, the resin was added to 12.5 g of Lys-OEt dihydrochloride pre-suspended in 250 ml chloroform and was equilibrated under mild stirring for 60 min at 4° C. The exchange resin was subsequently filtered off and the chloroform was evaporated at 10° C. using vacuum pump, yielding Lys-OEt in liquid state. The recovered resin was regenerated by washing with 200 ml of 1 M NaOH aqueous solution and reused another two times for repeating the above desalting procedure of Lys-OEt dihydrochloride. In total 23.9 g liquid Lys-OEt were obtained.

### Bulk Polymerization of Desalted Lys-OEt:

16.9 g of Lys-OEt was poured into a round bottom flask, and 2.54 g of Novozym 435 (CALB immobilized on polymeric resin, purchased from Novozymes), together with 1.69 ml of ethanol was added. The bulk polymerization was performed under stirring at 10° C. for 72 h. The crude product was characterized after filtration. The DP range was 2 to 25 with 6 at peak maximum, the conversion was 97% and the regioselectivity (ε) was 85%.

### Example 55

### Demineralization of Lys-OEt Dihydrochloride:

360 g of anion exchange resin type II Amberlite IRA410 (Cl form) purchased from Sigma Aldrich were washed with 360 ml of 1 M NaOH aqueous solution, in order to be converted to the OH form. The resin was further washed with 360 ml of deionized water, followed by 90 ml of ethanol. Then, the resin was added to 45 g of Lys-OEt dihydrochloride pre-suspended in 900 ml chloroform and was equilibrated under mild stirring for 60 min at 4° C. The exchange resin was subsequently filtered off and the chloroform was evaporated at 10° C. using vacuum pump, yielding Lys-OEt in liquid state. The recovered resin was regenerated by washing with 360 ml of 1 M NaOH aqueous solution and reused another three times for repeating the above desalting procedure of Lys-OEt dihydrochloride. In total 143 g liquid Lys-OEt were obtained.

### Bulk Polymerization of Desalted Lys-OEt:

15 μl of Lys-OEt was poured into a DSC pan, 2.3 mg of Novozym 435 (CALB immobilized on polymeric resin, purchased from Novozymes) was added and the pan was capped and sealed. The bulk polymerization proceeded at 10° C. for 60 h. The DP range was 2 to 39 with 7 at peak maximum and the regioselectivity (ε) was 94%.

### Example 56

### Demineralization of Lys-OEt Dihydrochloride:

100 g of Lys-OEt dihydrochloride was dissolved in 140 ml of 5 M NaOH aqueous solution in an ice bath. The resultant solution was mixed with 1 l of chloroform inside a jacketed glass vessel under vigorous stirring at 500 rpm at 10° C. for 15 min. The two phases were then allowed to separate without stirring for 15 min. The organic phase was collected and dried over anhydrous sodium sulfate. Chloroform was evaporated at 10° C. using vacuum pump, yielding Lys-OEt in liquid state. The extraction from the aqueous phase was repeated another two times using fresh chloroform.

### Bulk Polymerization of Desalted Lys-OEt:

290 ml of Lys-OEt was poured into a jacketed glass reactor equipped with a Rushton impeller, and 43.5 g of Novozym 435 (CALB immobilized on polymeric resin, purchased from Novozymes), together with 43.5 ml of ethanol was added. The bulk polymerization proceeded under stirring at 10° C. for 72 h. The crude product was characterized after filtration. The DP range was 2 to 25 with 5 at peak maximum, the conversion was 85% and the regioselectivity (8) was 88%.

### Example 57

Lys-OEt was desalted according to Example 10 and then polymerized in bulk in presence of immobilized trypsin, according to Example 22. The bulk polymerization was performed at 10° C. for 24 h, while crude samples were taken between 1 h and 24 h. The results from characterization of the crude samples are available in Table 6.

TABLE 6
Parameters from process in Example 57.

| | | | | Regioselectivity |
|---|---|---|---|---|
| Time | DP range | DPpeak max | Conversion | (α) |
| 1 h | 2-16 | 7 | 50 | 83 |
| 3 h | 2-21 | 7 | 68 | 79 |
| 6 h | 2-24 | 7 | 79 | 88 |
| 12 h | 2-28 | 8 | 86 | 93 |
| 24 h | 2-35 | 11 | 97 | 93 |

### Example 58

### Immobilization of Trypsin:

Trypsin was immobilized on polymeric resin ECR8209F (Purolite) according to the procedure in Example 33, with the exception that 15 g of resin ECR8209F were used. The immobilization yield was 8.4%. The enzyme activity was 0.447 U/g.

### Bulk Polymerization of Desalted Lys-OEt:

17.1 mL of Lys-OEt, desalted according to Example 25, was poured into a round bottom flask. Subsequently, 14.2 g of resin ECR8209F with trypsin immobilized according to the above procedure, was added to the flask. The bulk polymerization was performed under stirring at 10° C. for 96 h. The crude product was characterized after filtration. The DP range was 3 to 46 with 10 at peak maximum, the conversion was 78% and the regioselectivity (α) was 88%.

### Example 59

Lys-OEt dihydrochloride was desalted in situ according to Example 35. Subsequently, 71 mg of resin ECR8209F with trypsin immobilized according to Example 58 was added to the microtube. The bulk polymerization proceeded at 10° C. for 72 h. The crude product was characterized after filtration. The DP range was 2 to 29 with 6 at peak maximum, the conversion was 94% and the regioselectivity (α) was 88%.

### Example 60

### Immobilization of Trypsin:

Trypsin was immobilized on polymeric resin ECR8209F (Purolite) according to the procedure in Example 26, with the exception that after the slurry was mixed and equilibrated, the resin was washed with 0.5 M NaCl aqueous solution four times, followed by washing with water another two times. The immobilization yield was 68%. The enzyme activity was 0.928 U/g.

### Recycling of Immobilized Trypsin:

The recyclability of the immobilized trypsin was tested. 200 μL of Lys-OEt, desalted according to Example 25, was poured into a polypropylene microtube. Subsequently, 160 mg of resin ECR8209F with trypsin immobilized according to the above procedure, was added to the tube. The bulk polymerization was performed at 10° C. for 72 h. Following the initial polymerization, the product was washed with 1000 μl of water, centrifuged at 6000 rpm for 1 min and the supernatant containing the resultant polylysine was removed. This washing step was repeated three times. Afterwards, 200 μl of desalted Lys-OEt was added to initiate the next polymerization cycle. Two repeated polymerization cycles were conducted. The polymer from each cycle was characterized.

The results are available in Table 7.

TABLE 7
Parameters from process in Example 60.

| | | | | Regioselectivity |
|---|---|---|---|---|
| Recycle | DP range | DPpeak max | Conversion | (α) |
| 0 | 2-39 | 10 | 97 | 90 |
| 1 | 2-39 | 9 | 92 | 88 |
| 2 | 3-37 | 7 | 79 | 77 |

### Example 61

### Immobilization of Trypsin:

500 mg of polymeric resin ECR8209F (Purolite) was washed with water, with a resin:buffer ratio of 1:2 w/v and the excess liquid was filtered out. The washing was repeated 4 times. The washed resin was added to a 25 mg/ml solution of trypsin (from Porcine Pancreas, for biochemistry, Wako) in 1× phosphate buffer saline pH 7.4, with a resin:buffer ratio of 1:4 w/v. The slurry was gently mixed for 18 h and was left to equilibrate without mixing for another 20 h. Subsequently, the supernatant was collected and the resin was washed with 0.5 M NaCl aqueous solution ten times, followed by washing with water another two times. The immobilization yield was 50%. The enzyme activity was 0.959 U/g.

### Bulk Polymerization of Desalted Lys-OEt:

200 μL of Lys-OEt, desalted according to Example 25, was poured into a polypropylene microtube. Subsequently, 130 mg of resin ECR8209F with trypsin immobilized according to the above procedure, was added to the tube. The bulk polymerization proceeded at 10° C. for 72 h. The crude product was characterized after filtration. The DP range was 2 to 39 with 12 at peak maximum, the conversion was 97% and the regioselectivity (α) was 92%.

### Example 62

### Immobilization of Trypsin:

Trypsin was immobilized on polymeric resin ECR8209F (Purolite) according to the procedure in Example 61, with the exception that the resin:buffer ratio in the immobilization slurry was 1:10 w/v. The immobilization yield was 12%. The enzyme activity was 1.867 U/g.

### Bulk Polymerization of Desalted Lys-OEt:

200 μL of Lys-OEt, desalted according to Example 25, was poured into a polypropylene microtube. Subsequently, 77 mg of resin ECR8209F with trypsin immobilized according to the above procedure, was added to the tube. The bulk polymerization proceeded at 10° C. for 72 h. The crude product was characterized after filtration. The DP range was 2 to 39 with 12 at peak maximum, the conversion was 87% and the regioselectivity (α) was 78%.

### Example 63

### Immobilization of Trypsin:

Trypsin was immobilized on polymeric resin ECR8209F (Purolite) according to the procedure in Example 61, with the exception that the concentration of trypsin in the immobilization buffer was 6.25 mg/ml. The immobilization yield was 63%. The enzyme activity was 0.824 U/g.

### Bulk Polymerization of Desalted Lys-OEt:

200 μL of Lys-OEt, desalted according to Example 25, was poured into a polypropylene microtube. Subsequently, 175 mg of resin ECR8209F with trypsin immobilized according to the above procedure, was added to the tube. The bulk polymerization proceeded at 10° C. for 72 h. The crude product was characterized after filtration. The DP range was 2 to 39 with 8 at peak maximum, the conversion was 95% and the regioselectivity (α) was 89%.

### Example 64

200 μL of Lys-OEt, desalted according to Example 25, was poured into a polypropylene microtube. Subsequently, 10 mg of native trypsin (non-immobilized trypsin, from Porcine Pancreas, for biochemistry, Wako) was added to the tube. The activity of native trypsin was 4000 USP/mg. The bulk polymerization proceeded at 10° C. for 72 h. The crude product was characterized after filtration. The DP range was 2 to 39 with 9 at peak maximum, the conversion was 85% and the regioselectivity (α) was 80%.

### Example 65

The process was carried out according to Example 64, with the exception that 20 mg of native trypsin (non-immobilized trypsin) was used for the polymerization. The DP range was 2 to 39 with 9 at peak maximum, the conversion was 84% and the regioselectivity (α) was 86%.

### Example 66

Bulk Polymerization of Arg-OEt with In Situ Desalting:

200 mg of Arg-OEt dihydrochloride, was thoroughly mixed in a polypropylene microtube with 100 μL of triethylamine and 20 μl of water, at 10° C. Subsequently, 10 mg of native trypsin (non-immobilized trypsin 1:250, from Porcine Pancreas, for cell culture, Wako) was added to the tube, to start the chemoenzymatic polymerization. The activity of native trypsin was 920 USP/mg. The bulk polymerization proceeded at 10° C. for 72 h. The crude product was characterized. DP was between 2-8 with 3 at peak maximum, conversion was 70% and regioselectivity (α) was 100%.

### Example 67

Bulk Polymerization of Arg-OEt with In Situ Desalting:

Arg-OEt dihydrochloride was desalted in situ according to Example 66. Subsequently, 4 mg of native proteinase K (non-immobilized protease, recombinant, for biochemistry, from Fujifilm Wako) was added to the microtube. The activity of native proteinase K was 38 U/mg, where 1 U produces a peptide equivalent to 1 μmol of tyrosine as Folin colorant, with hemoglobin derived from bovine blood as substrate, per minute at 37° C. The bulk polymerization proceeded at 10° C. for 24 h. The crude product was characterized. DP was between 2-8 with 4 at peak maximum, conversion was 71% and regioselectivity (α) was 100%.

### Example 68

Bulk Polymerization of Arg-OEt with In Situ Desalting:

The process was carried out according to Example 67, with the exception that the polymerization was performed for 72 h. DP was between 2-9 with 4 at peak maximum. The regioselectivity (α) was 100%.

### Example 69

Bulk Polymerization of Arg-OEt with In Situ Desalting:

Arg-OEt dihydrochloride was desalted in situ according to Example 66. Subsequently, 4 mg of native α-chymotrypsin (from Bovine Pancreas, powder TCI) was added to the microtube. The activity of native α-chymotrypsin was 28 U/mg, where 1 U hydrolyzes 1.0 μmol of N-benzoyl-L-tyrosine ethyl ester per minute at 25° C., pH 7.8. The bulk polymerization was performed at 10° C. for 96 h, while crude samples were taken between 6 h and 96 h. The results from characterization of the crude samples are available in Table 8.

TABLE 8
Parameters from process in Example 69.

| | | | | Regioselectivity |
|---|---|---|---|---|
| Time | DP range | DPpeak max | Conversion | (α) |
| 6 | 2-17 | 6 | — | 100 |
| 15 | 2-20 | 7 | — | 100 |
| 24 | 2-22 | 6 | 74 | 100 |
| 96 | 2-22 | 9 | 78 | 100 |

### Example 70

Bulk Polymerization of Arg-OEt with In Situ Desalting:

200 mg of Arg-OEt dihydrochloride was thoroughly mixed in a polypropylene microtube with 50 μL of 5 M aqueous NaOH solution, at 10° C. Subsequently, 4 mg of native α-chymotrypsin (same as in Example 69) was added to the microtube. The bulk polymerization proceeded at 10° C. for 72 h. The crude product was characterized. DP was between 2-21 with 8 at peak maximum, conversion was 81% and regioselectivity (α) was 100%.

### Polymer Degradability

### Biodegradability in Presence of Wastewater Sludge

The biodegradability in presence of activated sludge was tested using freeze-dried polymers, following the OECD 301F biodegradation protocol. The results are shown in Table 9.

TABLE 9
Biodegradability under OECD 301F.

| | Biodegradability | Biodegradability |
|---|---|---|
| | level within 28 | level within 56 |
| Example | days (%) | days (%) |
| Ex. 42 | 62 | n.a. |
| Ex. 54 | 72 | 80 |
| Ex. 57 | 79 | 89 |
| Ex. 58 | 81 | 85 |

### Proteolytic Degradation in Presence of Trypsin

The degradability in presence of trypsin was investigated in an vitro proteolytic degradation assay. An aqueous solution of ε- or α-polylysine prepared according to Examples 42 and 57 accordingly (7.5 mg) in Milli-Q water (0.5 mL) was kept at 30° C. and a solution of trypsin (1 mg) in Milli-Q water (0.5 mL) was added. The final solution was stirred using ThermoMixer C (Eppendorf, Hamburg, Germany) at 30° C. and 1800 rpm for 12 h. As a control, the same reaction without trypsin was also performed. After the reaction, trypsin was removed by ultrafiltration using Vivaspin™ 500 (MWCO: 10,000) at 9000 rpm for 10 min, and the filtrate was lyophilized. The molecular weight distribution of the resulting solid was analyzed by gel permeation chromatography (GPC) using Nexera series with a system controller SCL-40 (Shimadzu, Kyoto, Japan). The sample (5 mg mL-1) was eluted with 0.5 M AcOH and 0.1 M NaNO3 aqueous solution at 40° C. and a flow rate of 1.0 mL min-1 using a Shodex column OHpak SB-803 HQ (Resonac, Tokyo, Japan). The sample was detected by a RI detector RID-20A and a photo diode array detector SPD-M40 (Shimadzu). The chromatogram was analyzed by LabSolutions software (Shimadzu) using poly(ethylene glycol) standards.

After the trypsin treatment, the GPC chromatogram including the peak top of ε-polylysine was almost unchanged. In contrast, the GPC chromatogram of α-polylysine showed a remarkable peak shift to lower molecular weight. This result clearly indicates that trypsin selectively cleaves the α-linked peptide bonds of polylysine, whereas the ε-linked peptide bond is not recognized in the catalytic pocket of trypsin.

1. A process for preparation of polylysine or polyarginine, wherein the process comprises chemoenzymatic polymerization of a lysine ester in bulk in the presence of an immobilized protease or a non-immobilized protease to provide α-polylysine regioselectively or in the presence of an immobilized lipase or a non-immobilized lipase to provide ε-polylysine regioselectively, or chemoenzymatic polymerization of an arginine ester in bulk in the presence of an immobilized protease or a non-immobilized protease to provide α-polyarginine regioselectively.

2. The process according to claim 1, wherein the lysine ester is selected from the group consisting of lysine alkyl esters, lysine C₁₋₆-alkyl esters, lysine aryl esters, lysine phenyl ester, lysine benzyl ester, lysine C₁₋₄-alkyl esters, lysine methyl ester, lysine ethyl ester, lysine propyl ester, and lysine butyl ester, and the arginine ester is selected from the group consisting of arginine alkyl esters, arginine C₁₋₆-alkyl esters, arginine aryl esters, arginine phenyl ester, arginine benzyl ester, arginine C₁₋₄-alkyl esters, arginine methyl ester, arginine ethyl ester, arginine propyl ester, and arginine butyl ester.

3. (canceled)

4. The process according to claim 1, wherein the protease is selected from the group consisting of serine proteases, trypsin, α-chymotrypsin, elastase, carboxypeptidase, subtilisin, cysteine proteases, papain, ficin, bromelain, analine, calpain, caspase, chymopapain, cathepsin, clostripain, and actinidin.

5. (canceled)

6. The process according to claim 1, wherein the lipase is selected from the group consisting of lipases (E.C. 3.1.1.3), a lipase from Candida antarctica B, Candida antarctica lipase B (CALB), a lipase from Aspergillus niger, Aspergillus niger lipase, a lipase from Burkholderia cepacia, Burkholderia cepacia lipase, a lipase from Rhizopus oryzae, and Rhizopus oryzae lipase.

7. (canceled)

8. The process according to claim 1, which further includes desalting a salt of the lysine ester to provide the lysine ester, or desalting a salt of the arginine ester to provide the arginine ester.

9. The process according to claim 1, wherein the chemoenzymatic polymerization is carried out at a temperature in the range of from −20° C. (minus 20° C.) to 80° C.

10. The process according to claim 1, wherein the chemoenzymatic polymerization is carried out for a period of at least 12 hours.

11. (canceled)

12. The process according to claim 1, which further includes separation of the immobilized enzyme or the non-immobilized enzyme from the polylysine or the polyarginine as obtained from the chemoenzymatic polymerization.

13. The process according to claim 12, which further includes applying the immobilized enzyme or the non-immobilized enzyme from the separation to a further chemoenzymatic polymerization of a lysine ester or an arginine ester.

14. The process according to claim 1, wherein the polylysine has a degree of polymerization in the range from 2 to 60, and the polyarginine has a degree of polymerization in the range from 2 to 30.

15. The process according to claim 14, wherein the range of the degree of polymerization of the polylysine has a peak maximum in the range of from 4 to 25, and the range of the degree of polymerization of the polyarginine has a peak maximum in the range of from 2 to 5.

16. A method for controlling regioselectivity of linear polylysine or polyarginine in the chemoenzymatic polymerization of a lysine ester or an arginine ester in bulk respectively, wherein an immobilized protease or a non-immobilized protease is used to provide α-polylysine or α-polyarginine regioselectively, or an immobilized lipase or a non-immobilized lipase is used to provide ε-polylysine regioselectively.

17. The method according to claim 16, wherein the lysine ester is selected from the group consisting of lysine alkyl esters, lysine C₁₋₆-alkyl esters, lysine aryl esters, lysine phenyl ester, lysine benzyl ester, lysine C₁₋₄-alkyl esters, lysine methyl ester, lysine ethyl ester, lysine propyl ester, and lysine butyl ester, and the arginine ester is selected from the group consisting of arginine alkyl esters, arginine C₁₋₆-alkyl esters, arginine aryl esters, arginine phenyl ester, arginine benzyl ester, arginine C₁₋₄-alkyl esters, arginine methyl ester, arginine ethyl ester, arginine propyl ester, and arginine butyl ester.

18. (canceled)

19. The method according to claim 16, wherein the protease is selected from the group consisting of serine proteases, trypsin, α-chymotrypsin, elastase, carboxypeptidase, subtilisin, cysteine proteases, papain, ficin, bromelain, analain, calpain, caspase, chymopapain, cathepsin, clostripain, and actinidin.

20. (canceled)

21. The method according to claim 16, wherein the lipase is selected from the group consisting of lipases (E.C. 3.1.1.3), a lipase from Candida antarctica B, Candida antarctica lipase B (CALB), a lipase from Aspergillus niger, Aspergillus niger lipase, a lipase from Burkholderia cepacia, Burkholderia cepacia lipase, a lipase from Rhizopus oryzae, and Rhizopus oryzae lipase.

22. (canceled)

23. The method according to claim 16, wherein the chemoenzymatic polymerization is carried out at a temperature in the range of from −20° C. to 80° C.

24. The method according to claim 16, wherein the chemoenzymatic polymerization is carried out for a period of at least 12 hours.

25. The method according to claim 16, wherein the regioselectivity of α-polylysine is at least 60%.

26. The method according to claim 16, wherein the regioselectivity of ε-polylysine is at least 70%.

27. The method according to claim 16, wherein the regioselectivity of ε-polyarginine is at least 75%.
